(19) United States
(12) Patent Application Publication
(10) Pub. No.: US 2010/0136124 A1
(43) Pub. Date: Jun. 3, 2010

(54) NANOPARTICLE-COATED CAPSULE FORMULATION FOR DERMAL DRUG DELIVERY

(75) Inventors: Clive Allan Prestidge, Semaphore South (AU); Spomenka Simovic, Adelaide (AU); Nasrin Ghouchi Eskandar, Prospect (AU)

Correspondence Address: ROTHWELL, FIGG, ERNST & MANBECK, P.C., 1425 K STREET, N.W., SUITE 800, WASHINGTON, DC, 20005, US

(73) Assignee: UNIVERSITY OF SOUTH AUSTRALIA, Adelaide, South Australia (AU)

(21) Appl. No.: 12/596,679

(22) PCT Filed: Apr. 21, 2008
(86) PCT No.: PCT/AU08/00555

§ 371 Date: Jan. 20, 2010

(30) Foreign Application Priority Data
Apr. 20, 2007 (AU) 2007902112

(51) Int. Cl. A61K 9/50; A61K 31/07; A61K 31/473; A61P 17/02

(52) U.S. Cl. 424/490; 514/725; 514/297; 977/906

(57) ABSTRACT

A method and formulation for the delivery of an active substance to the skin (epidermis, including the stratum corneum and viable epidermis, and dermis) of a subject. The formulation comprises oil-based or aqueous droplets comprising the active substance within a coating of nanoparticles, particularly silica nanoparticles. The active substance may be suitable for the treatment of a disease or condition which is localised, or at least partially localised, to the skin (eg skin cancer, psoriasis, eczema, infections including bacterial and fungal infections, acne, dermatitis, inflammation, and rheumatoid arthritis).

### FIELD OF THE INVENTION

The present invention relates to a method and formulation for the delivery of an active substance to the skin (epidermis, including the stratum corneum and viable epidermis, and dermis) of a subject. The formulation comprises oil-based or aqueous droplets comprising the active substance within a coating of nanoparticles, particularly silica nanoparticles.

### INCORPORATION BY REFERENCE

This patent application claims priority from:
AU 2007902112 titled “Nanoparticle-coated capsule formulation for dermal delivery” filed on 20 Apr. 2007.

The entire content of this application is hereby incorporated by reference.

The following international patent applications are referred to herein:
PCT/AU2006/000771 (WO 2006/130904) titled “Dried formulations of Nanoparticle-coated capsules”, and
PCT/AU2007/000602 (WO 2007/128066) titled “Drug release from nanoparticle-coated capsules”.

The entire content of both of these applications is also hereby incorporated by reference.

### BACKGROUND OF THE INVENTION

Delivery of active substances to skin, such as human skin, has the potential to be a particularly useful way of locally delivering active substances such as those having cosmetic and/or therapeutic significance. However, delivery of active substances to the skin poses a problem due to the natural protective barrier function of skin. That is, the structure of skin is such that it naturally protects the body from the entrance of foreign material such as microorganisms and chemicals, and from the loss of endogenous material such as water. Skin has a multi-layered structure, with each layer of skin representing different levels of cellular or epidermal differentiation. The epidermis is the outer most layer, which consists primarily of layers of keratinised epithelium, under which lies the dermis, a layer of connective tissue which contains a rich network of blood and lymph vessels, hair follicles and sweat and sebaceous glands. The external layer of the epidermis is the stratum corneum (SC). This layer is relatively impermeable to water; having a lipophilic nature that primarily accounts for the barrier nature of skin (Elias P. M., 1983). The stratum corneum does, however, show selective permeability in that it permits relatively lipophilic compounds to diffuse into the lower layers, primarily by passive transportation (Scheuplein R. J. and Blank I. H, 1971).

Dermal delivery is the delivery of an agent (eg an active substance) to the skin (epidermis, including stratum corneum, and dermis) via topical application to the skin surface. In contrast, transdermal delivery is the delivery of an agent (eg an active substance), again via topical application to the skin surface, but in this case through the various layers of the dermis and into the systemic circulation.

Dermal delivery of an active substance may be desired, for example, when targeting sites within the skin in situations where minimal or no transfer to the systemic circulation is required (eg to treat diseases and conditions which are localised, or at least partially localised, to the skin, such as skin cancer, psoriasis, eczema, microbial infections including fungal infections, and acne). It is also desirable to deliver many cosmetic and therapeutic substances dermally, for example, the active substance(s) in anti-wrinkle and/or anti-ageing creams. Dermal delivery via topical application of an active substance to the skin surface therefore provides an advantage over several other delivery techniques in that it allows for the direct targeting of a site of interest, and is generally considered as being “non-invasive” which offers improved patient acceptance, compliance and ease of application.

However, previous attempts to deliver various active substances to the dermis by topical application to the skin surface have not been widely successful, generally because the active substance has not sufficiently penetrated through the epidermis. Indeed, it has been shown that molecules must be less than 500 Da to pass through the SC (Bos J. D. and Meinardi M. M. H. M., 2000; and Brown M. B., et al., 2006) and must, additionally, have a suitable aqueous and lipid solubility. Accordingly, topical application of an active substance does not ensure its delivery to the dermis.

A variety of methods have been shown to enhance skin permeability, including electrical methods (eg iontophoresis and electroporation), mechanical methods (eg microneedle, puncture, perforation, abrasion, suction and stretching) as well as other methods including ultrasound, magnetophoresis, and thermophoresis. However, the efficacy of these methods is variable, and in many cases, the invasive nature of some of the methods makes them undesirable.

Accordingly, there is a need for the development of new methods and formulations for the delivery of an active substance to the dermis of a subject by topical application.

### SUMMARY OF THE INVENTION

In a first aspect, the present invention provides a method of dermal delivery of an active substance, said method comprising topically applying to the skin of a subject a formulation comprising droplets of a suitable carrier comprising said active substance and, optionally, an emulsifier, wherein said droplets are coated on their surface with at least one layer of nanoparticles, and wherein said active substance is not retinol or a retinol derivative.

In a second aspect, the present invention provides a formulation for topical application to the skin, wherein said formulation comprises droplets of a suitable carrier comprising an active substance and, optionally, an emulsifier, wherein said droplets are coated on their surface with at least one layer of nanoparticles, and wherein said active substance is not retinol or a retinol derivative.

The formulation of the present invention may release the active substance in a controlled manner, for example, in a sustained manner or, otherwise, such that the active substance is rapidly released upon application to the skin surface.

The active substance may be suitable for the treatment of a disease or condition which is localised, or at least partially localised, to the skin, such as skin cancer, psoriasis, eczema, infections including bacterial and fungal infections, acne, dermatitis, inflammation, and rheumatoid arthritis. Thus, for example, for treatment of skin cancer (eg small basal cell carcinomas and solar keratoses), the active substance may be selected from chemotherapy agents, particularly 5-fluorouracil.

Alternatively, the active substance may be selected from active ingredients commonly included in cosmetics such as anti-wrinkle and/or anti-ageing creams, or sunscreens. Thus, the active substance might therefore be selected from tocopherols (vitamin E), coenzyme Q10 (ubiquinone), UV-A absorbers (eg avobenzene) and UV-B absorbers (eg octyl methoxycinnamate), titanium dioxide and zinc oxide.

### BRIEF DESCRIPTION OF THE FIGURES

FIG. 1 provides a graph showing the degradation kinetics of vitamin A (retinol) contained in negatively charged nanoparticle-coated capsules, wherein the emulsion is stablised by lecithin (▪ lecithin stabilised bare emulsion (L); lecithin stabilised emulsion with silica in oil phase (LSO); ▴ lecithin stabilised bare emulsion with silica in water phase (LSA); and ▾ oil in water emulsion (O/W));

FIG. 2 provides a graph showing the degradation kinetics of vitamin A (retinol) contained in positively charged nanoparticle-coated capsules, wherein the emulsion is stablised by oleylamine (▪ oleylamine stabilised bare emulsion (O); oleylamine stabilised emulsion with silica in oil phase (OSO); ▴ oleylamine stabilised emulsion with silica in water phase (OSA); and ▾ oil in water emulsion (O/W));

FIG. 3 provides a graph of the release profile of vitamin A (retinol) from negatively charged nanoparticle-coated capsules (▪ lecithin stabilised bare emulsion (L); lecithin stabilised emulsion with silica in oil phase (LSO); and ▴ lecithin stabilised emulsion with silica in water phase (LSA));

FIG. 4 provides a graph of the release profile of vitamin A (retinol) from positively charged nanoparticle-coated capsules (▪ oleylamine stabilised bare emulsion (O); oleylamine stabilised emulsion with silica in oil phase (OSO); ▴ oleylamine stabilised emulsion with silica in water phase (OSA));

FIG. 5 provides a graph showing the retention of vitamin A (retinol) in pig skin samples over 24 hours from a lecithin-stabilised formulation of the present invention (L=lecithin-stabilised emulsion of all-trans-retinol in a triglyceride oil; LSO=lecithin-stabilised nanoparticle coated emulsion of all-trans-retinol in a triglyceride oil, wherein the capsules were formed from a mix with the nanoparticles provided in the oil phase; and LSA=lecithin-stabilised nanoparticle coated emulsion of all-trans-retinol in a triglyceride oil, wherein the capsules were formed from a mix with the nanoparticles provided in the aqueous phase);

FIG. 6 provides a graph showing the penetration of vitamin A (retinol) through pig skin samples from a lecithin-stabilised formulation of the present invention (L=lecithin-stabilised emulsion of all-trans-retinol in a triglyceride oil; LSO=lecithin-stabilised nanoparticle coated emulsion of all-trans-retinol in a triglyceride oil, wherein the capsules were formed from a mix with the nanoparticles in the oil phase; and LSA=lecithin-stabilised nanoparticle coated emulsion of all-trans-retinol in a triglyceride oil, wherein the capsules were formed from a mix with the nanoparticles in the aqueous phase);

FIG. 7 provides a graph showing the retention of vitamin A (retinol) in pig skin samples over 24 hours from an oleylamine-stabilised formulation of the present invention (O=oleylamine-stabilised emulsion of all-trans-retinol in a triglyceride oil; OSO=oleylamine-stabilised nanoparticle coated emulsion of all-trans-retinol in a triglyceride oil, wherein the capsules were formed from a mix with the nanoparticles in the oil phase; and OSA=oleylamine-stabilised nanoparticle coated emulsion of all-trans-retinol in a triglyceride oil, wherein the capsules were formed from a mix with the nanoparticles in the aqueous phase); and

FIG. 8 provides a graph showing the penetration of vitamin A (retinol) through pig skin samples from an oleylamine-stabilised formulation of the present invention (O=oleylamine-stabilised emulsion of all-trans-retinol in a triglyceride oil; OSO=oleylamine-stabilised nanoparticle coated emulsion of all-trans-retinol in a triglyceride oil, wherein the capsules were formed from a mix with the nanoparticles in the oil phase; and OSA=oleylamine-stabilised nanoparticle coated emulsion of all-trans-retinol in a triglyceride oil, wherein the capsules were formed from a mix with the nanoparticles in the aqueous phase); and

FIG. 9 provides a graph showing the distribution of vitamin A (retinol) in pig skin samples treated with a lecithin-stabilised formulation of the present invention (L=lecithin-stabilised emulsion of all-trans-retinol in a triglyceride oil; LSO=lecithin-stabilised nanoparticle coated emulsion of all-trans-retinol in a triglyceride oil, wherein the capsules were formed from a mix with the nanoparticles in the oil phase; and LSA=lecithin-stabilised nanoparticle coated emulsion of all-trans-retinol in a triglyceride oil, wherein the capsules were formed from a mix with the nanoparticles in the aqueous phase); and

FIG. 10 provides a graph showing the distribution of vitamin A (retinol) in pig skin samples treated with an oleylamine-stabilised formulation of the present invention (O=oleylamine-stabilised emulsion of all-trans-retinol in a triglyceride oil; OSO=oleylamine-stabilised nanoparticle coated emulsion of all-trans-retinol in a triglyceride oil, wherein the capsules were formed from a mix with the nanoparticles in the oil phase; and OSA=oleylamine-stabilised nanoparticle coated emulsion of all-trans-retinol in a triglyceride oil, wherein the capsules were formed from a mix with the nanoparticles in the aqueous phase).

### DETAILED DESCRIPTION OF THE INVENTION

In a first aspect, the present invention provides a method of dermal delivery of an active substance, said method comprising topically applying to the skin of a subject a formulation comprising droplets of a suitable carrier comprising said active substance and, optionally, an emulsifier, wherein said droplets are coated on their surface with at least one layer of nanoparticles, and wherein said active substance is not retinol or a retinol derivative.

The active substance may be delivered to the skin, including the stratum corneum, the other layers of the epidermis and the dermis. In a preferred embodiment, the active substance is delivered primarily to the dermis.

In a second aspect, the present invention provides a formulation for topical application to the skin, wherein said formulation comprises droplets of a suitable carrier comprising an active substance and, optionally, an emulsifier, wherein said droplets are coated on their surface with at least one layer of nanoparticles, and wherein said active substance is not retinol or a retinol derivative.

The formulation of the present invention may release the active substance in a controlled manner, for example, in a sustained manner or, otherwise, such that the active substance is rapidly released upon application to the skin surface.

A formulation according to the present invention may be produced by, for example, any of the suitable methods described in international patent application Nos PCT/AU2006/000771 (WO 2006/130904) and PCT/AU2007/000602 (WO 2007/128066).

More particularly, a formulation according to the present invention, which upon application to the skin, is capable of releasing the active substance in a sustained manner, may be produced by a method comprising the following steps:
(i) dispersing a discontinuous phase comprising a suitable carrier and an active substance into a continuous phase so as to form a two-phase liquid system comprising droplets of said discontinuous phase, each of said droplets having, at its surface, a phase interface; and
(ii) allowing nanoparticles provided to said two-phase liquid system to congregate at the phase interface to thereby coat said surface of the droplets in at least one layer of said nanoparticles;
   wherein said two-phase liquid system is formed, or is otherwise adjusted, so as to have a concentration of a suitable electrolyte which enhances the nanoparticle congregation of step (ii) such that the coating on said surface of the droplets provided by the at least one layer of said nanoparticles presents a semi-permeable barrier to the active substance.

On the other hand, a formulation according to the present invention, which upon application to the skin, is capable of releasing the active substance in a rapid manner, may be produced by a method comprising the following steps:
(i) dispersing a discontinuous phase comprising a suitable carrier and an active substance into a continuous phase so as to form a two-phase liquid system comprising droplets of said discontinuous phase, and each of said droplets having, at its surface, a phase interface; and
(ii) allowing nanoparticles provided to said two-phase liquid system to congregate at the phase interface to thereby coat said surface of the droplets in at least one layer of said nanoparticles to form a nanoparticle-coated capsule formulation;
   wherein the active substance is present in the discontinuous phase in an amount greater than its solubility limit in the discontinuous phase.

Preferably, the discontinuous phase is an oil-based or lipidic medium carrier and the continuous phase is aqueous. Alternatively, the discontinuous phase is an aqueous carrier and the continuous phase is an oil-based or lipidic medium. In a particular embodiment of the latter, the discontinuous phase is an aqueous carrier and each droplet is surrounded by a single or multiple lipid bilayer (ie thereby forming a liposome), and the continuous phase is aqueous.

Suitable aqueous carriers include water or polymer dispersions, while suitable oil-based or lipidic medium carriers include triglyceride oils, medium chain triglycerides, paraffin oil, soybean oil, and jojoba oil.

The active substance may be selected from nutriceutical substances, cosmetic substances (including sunscreen agents), and drug compounds. More than one active substance (eg for combination therapies) may be included in a formulation according to the present invention.

Accordingly, the active substance may be selected so as to treat a disease or condition which is localised, or at least partially localised, to the skin, such as skin cancer, psoriasis, eczema, infections including bacterial and fungal infections, acne, inflammation, rheumatoid arthritis and dermatitis. Thus, for treatment of skin cancer (eg small basal cell carcinomas and solar keratoses), the active substance may be selected from chemotherapy agents, particularly 5-fluorouracil. For treatment of psoriasis, the active substance may be selected from vitamin D and analogues thereof, corticosteroids, anthralin, cyclosporin A, and combinations thereof. In the case of eczema, the active substance may be selected from corticosteroids, and immunomodulatory compounds such as pimecrolimus and tacrolimus, and combinations thereof. For treatment of infections, the active substance may be selected from antibiotic agents (eg benzoyl peroxide, clindamycin, erythromycin, tetracycline, and combinations thereof) and antifungal agents (eg imidazole compounds, thiocarbamate compounds, allylamine, and combinations thereof), while for the treatment of inflammation and rheumatoid arthritis, the active substance may be selected from non-steroidal anti-inflammatory drugs (eg celecoxib, diclofenac, indomethacin, piroxicam, ketoprofen, ibuprofen, and naproxen) and steroidal anti-inflammatory drugs (eg prednisone, prednisolon, and hydrocortisone) and local anaesthetics (eg lidocain, lidocain-prilocaine eutectic mixtures). Where the active substance is an antibiotic agent or tretinoin, the formulation may be suitable for the treatment of acne.

Alternatively, the active substance may be selected from active ingredients commonly included in cosmetics such as anti-wrinkle and/or anti-ageing creams, or sunscreens. Thus, the active substance might therefore be selected from tocopherols (vitamin E), coenzyme Q10 (ubiquinone), UV-A absorbers (eg avobenzene) and UV-B absorbers (eg octyl methoxycinnamate), titanium dioxide and zinc oxide.

The active substance will typically be present in the discontinuous phase at a concentration in the range of 0.01 to 10 wt %, however, it will be well recognised by persons skilled in the art that the actual amount present may vary considerably depending upon, for example, the solubility of the particular active substance (which can often be increased by the presence of an emulsifier in the discontinuous phase or by otherwise initially providing the nanoparticles in the discontinuous phase) and the manner of release of the active substance that is desired (ie for a rapid release formulation, the active substance may be present in an amount that is greater than its solubility limit in the discontinuous phase, and will therefore preferably be present in an amount that is at least about 110%, more preferably at least about 120%, of the solubility limit of the active substance in the discontinuous phase).

The nanoparticles may be hydrophilic or hydrophobic. In one preferred embodiment, the droplets will be coated with a single layer, or multiple layers, of hydrophilic or hydrophobic nanoparticles. However, in another preferred embodiment, the droplets will be coated with at least two layers of nanoparticles, with the inner layer comprised of hydrophobic nanoparticles and the outer layer comprised of hydrophilic nanoparticles.

Preferably, said nanoparticles have an average diameter of 5-2000 nm, more preferably, 20-80 nm, most preferably about 50 nm. Also, preferably, the size of the nanoparticles will be such that the ratio of nanoparticle size to capsule size (ie the size of the encapsulated droplets) does not exceed 1:15.

Preferably, the nanoparticles are silica nanoparticles, however nanoparticles composed of other substances (eg titania and latex) are also suitable.

Optionally, an emulsifier can be used to stabilise the droplets prior to the congregation of the nanoparticles onto the surfaces of the droplets. Suitable emulsifiers include lecithin, oleylamine, sodium deoxycholate, 1,2-distearyl-sn-glycero-3-phosphatidyl ethanolamine-N, stearylamine and 1,2-dioleoyl-3-trimethylammonium-propane. However, typically any emulsifier that has a HLB (hydrophilic-lipophilic balance) value of less than about 12 can be used. On the other hand, hydrophilic emulsifiers such as sodium dodecyl sulphate (SDS) are less suitable, since these can readily migrate into the continuous phase where they can coat both the droplets and the nanoparticles, when present in high concentrations, thereby preventing nanoparticle congregation.

Preferred emulsifiers are lecithin (which confers a negative charge to the droplets) and oleylamine (which confers a positive charge to the droplets). Most preferred, is oleylamine.

The emulsifier will typically be provided in an amount in the range of 0.0001 to 10 wt %, more preferably, in the range of 0.01 to 1 wt %.

In some embodiments, the emulsifier can have a significant effect on the stability of the active substance. For example, the emulsifier may reduce degradation and/or increase the half life of the active substance.

Preferably, a formulation according to the present invention will be produced in the presence of an amount of electrolyte (eg NaCl and/or KNO₃) suitable to enhance the congregation of the nanoparticles at the phase interface.

The amount of the electrolyte will typically be at least 0.5×10⁻⁴ M, although a lesser concentration of electrolyte may, however, suffice (eg 1×10⁻⁶ to 1×10⁻⁵ M). Preferably, the amount of electrolyte will be at least 1×10⁻³ M, but no more than 1×10⁻¹ M.

For a formulation capable of releasing the active substance in a sustained manner, the formulation will preferably be formed from a two-phase liquid system that has been formed, or is otherwise adjusted, so as to have a concentration of a suitable electrolyte which enhances the nanoparticle congregation such that the coating on said surface of the droplets (ie the coating provided by the at least one layer of said nanoparticles) presents a semi-permeable barrier to the active substance. By “semi-permeable barrier”, it is to be understood that the coating substantially retards the diffusion of the active substance from within the encapsulated droplets, such that the active substance is released in a controlled manner, in particular, in a sustained manner. Preferably, the semi-permeable barrier presented by the nanoparticle coating retards the diffusion of the active substance from within the encapsulated droplets such that after two hours of being placed in a test medium (eg MilliQ water), at least 25% of the active substance content of the encapsulated droplets has been retained within the encapsulated droplets (ie no more than 75% of the active substance content has been released into the test medium). More preferably, the semi-permeable barrier retards the diffusion of the active substance content of the encapsulated droplets such that at least 35%, and most preferably at least 45%, of the active substance has been retained within the encapsulated droplets after two hours of being placed in a test medium.

Optionally, the encapsulated droplets are provided with a polymer layer around the periphery to modify the interfacial properties of the capsule. Such a polymer layer may comprise cellulose derivatives such as hydroxypropylmethylcellulose and chitosan, or a carbomer, or a mixture thereof.

The discontinuous phase may, optionally, be cross-linked or otherwise further comprise a gelling material so as to form a matrix. Such a matrix may enhance the controlled release of an active substance (ie sustained release) from the encapsulated droplets.

A formulation according to the present invention may be reconstituted from a dried formulation (ie the encapsulated droplets (capsules) of the dried formulation may be re-dispersed into a liquid to re-form a two-phase liquid system). Methods for producing dried nanoparticle-coated capsule formulation are described in international patent application No PCT/AU2006/000771 (WO 2006/130904). Such methods include drying with a rotary evaporator, freeze drying, spray drying or drying using fluidised bed procedures or pressure filtration coupled with vacuum drying.

A formulation according to the present invention may constitute or comprise a coacervate of nanoparticle-coated capsules.

Further, a formulation according to the present invention may further comprise other agents and substances such as thickening agents, preservatives, antioxidants, fragrances, colour stabilisers, pH stabilisers and moisturisers that are commonly found in formulations for topical application.

In order that the nature of the present invention may be more clearly understood, preferred forms thereof will now be described with reference to the following non-limiting examples.

### EXAMPLES

### Example 1

### Preparation of Vitamin A Nanoparticle-Coated Capsule Formulation

Retinol (vitamin A alcohol) was used as a model active substance. It is an active substance of considerable interest to the pharmaceutical, nutritional and cosmetic industries, however formulating the substance has previously been met with difficulties due to its sensitivity to oxidation (eg photo-oxidation upon exposure to light). In particular, retinol is sensitive to auto-oxidation at the unsaturated side-chain of the compound, resulting in the formation of decomposition products, isomerisation and polymerisation. As a result, auto-oxidation leads to reduced biological activity, and an increased risk of toxicity caused through generation of decomposition products. A nanoparticle stabilised emulsion of retinol was produced to first assess whether such a formulation could enhance the stability of the retinol and satisfactorily release the retinol to a desired site.

### a) Preparation of Vitamin A-Containing Emulsion Stabilised by Lecithin

Lecithin (0.6 g) emulsifier and all-trans-retinol (0.05 g) was dissolved in triglyceride oil (Miglyol 812™) (10 g), and then added to water (total sample weight: 100 g) for control emulsions, or to the silica dispersion described in step (c), to form capsules as described in step (d) below. In some experiments, the emulsifier, retinol and oil mixture was added to water and a portion of the water was replaced with the silica dispersion described in step (c), to form capsules as described in step (d) below. The resulting product was mixed using a high pressure homogeniser (5 cycles at 500 to 1000 bars). The concentration of electrolyte of the two-phase liquid system was estimated to be within the range of about 1×10⁻⁶ to 1×10⁻⁵ M (NaCl). No additional electrolyte was added.

### b) Preparation of Vitamin A-Containing Emulsion Stabilised by Oleylamine

Oleylamine (1 g) emulsifier and all-trans-retinol (0.05 g) was dissolved in triglyceride oil (Miglyol 812™) (10 g), and then added to water (total sample weight: 100 g) for control emulsions, or to the silica dispersion described in step (c), to form capsules as described in step (d) below. In some experiments, the emulsifier, retinol and oil mixture was added to water and a portion of the water was replaced with the silica dispersion described in step (c), to form capsules as described in step (d) below. The resulting product was mixed using a high pressure homogeniser (5 cycles at 500 to 1000 bars). The concentration of electrolyte of the two-phase liquid system was estimated to be within the range of about 1×10⁻⁶ to 1×10⁻⁵M (NaCl). No additional electrolyte was added.

### c) Preparation of Nanoparticles

An aqueous dispersion of fumed silica (Aerosil® 380) nanoparticles (1 wt %) (ie hydrophilic nanoparticles) was prepared by sonication over at least a one hour period.

### d) Capsule Formation

For emulsions containing silica nanoparticles initially included in the aqueous phase, capsules were formed when the nanoparticle dispersion of step (c) was separately mixed with either of the emulsions as described in step (a) and step (b).

### e) Alternative Capsule Preparation (Silica Nanoparticles in Oil)

Capsules were also formed in an analogous manner wherein the nanoparticles are initially included in the triglyceride oil (ie silica in oil formulations) from which the emulsion is formed. That is, lecithin-stabilised nanoparticle-coated retinol capsules, similar to those described in (a) above, were prepared by dissolving lecithin (0.6 g) emulsifier in the triglyceride oil (Miglyol 812™) (10 g) to which fumed silica (Aerosil® 380) nanoparticles (1 wt %) were then added. Then, all-trans-retinol (0.05 g) was dissolved in the triglyceride oil mixture and water was added (total sample weight: 100 g). An emulsion was formed using a high pressure homogeniser (5 cycles at 500 to 1000 bars).

Further, oleylamine-stabilised nanoparticle-coated retinol capsules, similar to those described in (b) above, except that nanoparticles were added directly to the triglyceride oil, were formed. Oleylamine (1 g) emulsifier was dissolved in the triglyceride oil (Miglyol 812™) (10 g) to which fumed silica (Aerosil® 380) nanoparticles (1 wt %) were then added. Then, all-trans-retinol (0.05 g) was dissolved in the triglyceride oil mixture and water was added (total sample weight: 100 g). An emulsion was formed using a high pressure homogeniser (5 cycles at 500 to 1000 bars).

### f) Capsule Characteristics

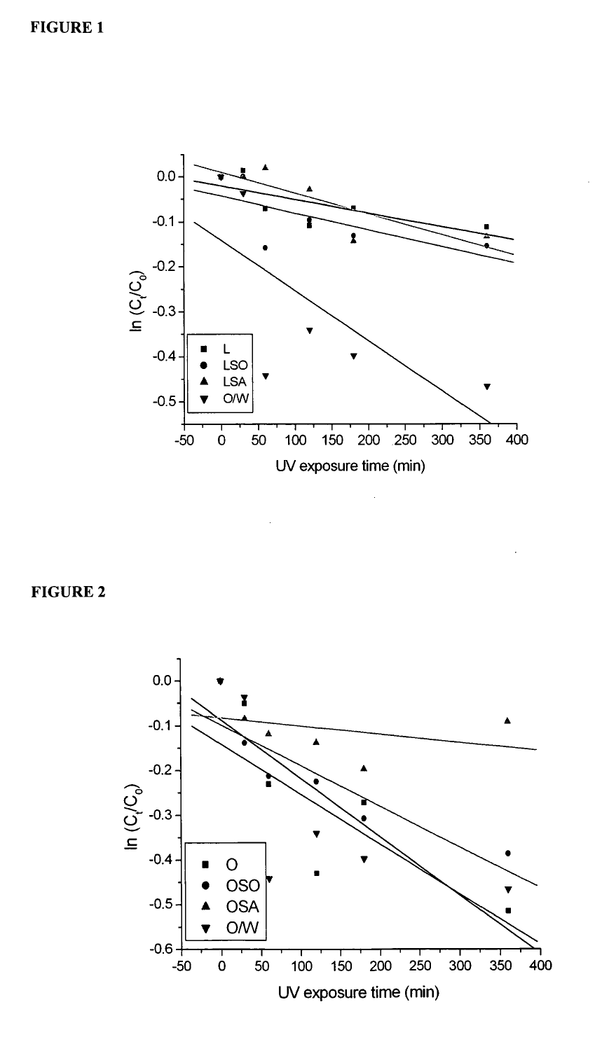

The capsules were assessed for stability of the retinol upon exposure to ultraviolet light (UVA+UVB) for up to 6 hours. The results are shown in FIGS. 1 and 2. The positively charged nanoparticle-coated capsules (ie capsules stabilised with oleylamine) showed particularly good stability against UV exposure. While not wishing to be bound by theory, it is considered that the less pronounced results for the negatively charged nanoparticle-coated capsules (ie capsules stabilised with lecithin) may have been due to a stabilising effect conferred by the lecithin per se on the retinol.

The capsules were also assessed for in vitro release of the active substance (ie retinol) using Franz diffusion cells with artificial cellulose membranes as follows. Membranes were pre-soaked in isopropyl myristate for 2 hours, and then the membrane was mounted on a Franz diffusion cell, using 5 ml of water-ethanol 50-50 as a receptor medium. 100 μL of the emulsion was added on the membrane surface. At determined time intervals, 200 μL of the “receptor phase” (that is, the phase that has passed through the membrane) is sampled and analysed by HPLC.

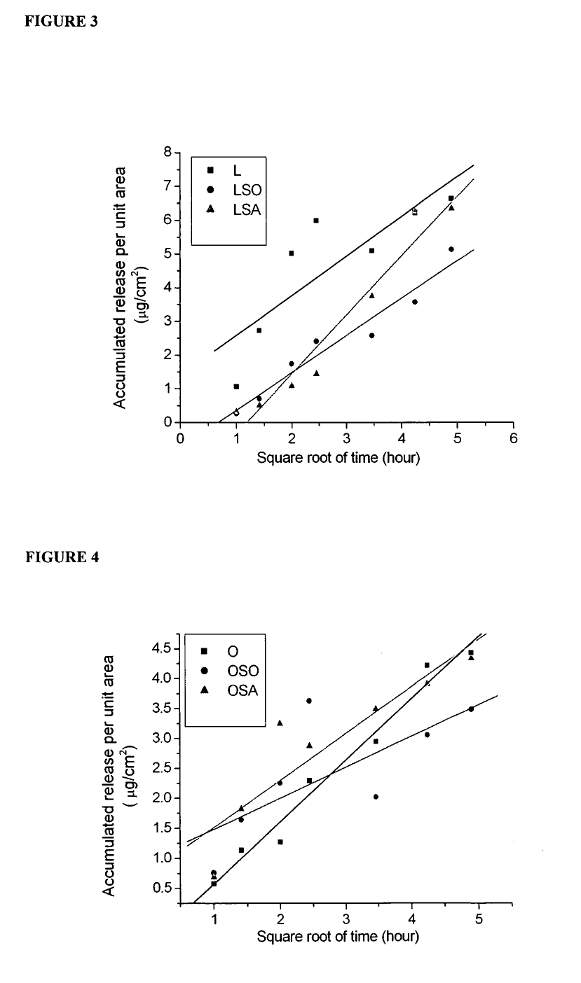

The analysis of the release profiles obtained (shown at FIGS. 3 and 4) showed that Higuchi's model is the most suitable for describing the release kinetics of the retinol:

Qₜ=K_{H}t^{1/2}

where
Q: the amount of drug released in time t per unit area
K_{H}: Higuchi's rate constant;
and the calculation of diffusion rate constants (see Table 1) from the slope of the line in the plot of released amount of drug per unit area of the membrane versus √t showed that the diffusion rate constant in the presence of silica nanoparticles decreased for both negatively and positively charged emulsions (ie the nanoparticle-coated capsules showed a sustained rate of retinol release). Thus, the nanoparticle-coated retinol capsule formulations increased the chemical stability of the retinol and were able to satisfactorily sustain the diffusion of retinol.

TABLE 1
Correlation of diffusion rate constant for the diffusion of drug from
different formulations

| | Rate Constant | |
|---|---|---|
| Formulation | (μg/cm²/h^{1/2}) | Correlation Coefficient |
| O/W | 0.88 | 0.9948 |
| L | 1.85 | 0.8690 |
| LSO | 1.10 | 0.9835 |
| LSA | 0.84 | 0.9598 |
| O | 1.07 | 0.9974 |
| OSO | 0.64 | 0.8871 |
| OSA | 0.92 | 0.9802 |

wherein:
O/W = oil in water;
L = lecithin-stabilised emulsion of all-trans-retinol in a triglyceride oil;
LSO = lecithin-stabilised nanoparticle coated emulsion of all-trans-retinol in a triglyceride oil, wherein the capsules were formed from a mix with the nanoparticles in the oil phase;
LSA = lecithin-stabilised nanoparticle coated emulsion of all-trans-retinol in a triglyceride oil, wherein the capsules were formed from a mix with the nanoparticles in the aqueous phase
O = oleylamine-stabilised emulsion of all-trans-retinol in a triglyceride oil;
OSO = oleylamine-stabilised nanoparticle coated emulsion of all-trans-retinol in a triglyceride oil, wherein the capsules were formed from a mix with the nanoparticles in the oil phase; and
OSA = oleylamine-stabilised nanoparticle coated emulsion of all-trans-retinol in a triglyceride oil, wherein the capsules were formed from a mix with the nanoparticles in the aqueous phase.

### Example 2

### Ex Vivo Dermal Delivery of Vitamin A from Nanoparticle-Coated Capsule Formulation

### a) Lecithin-Stabilised Formulations (Negatively Charged Capsules)

A study of the release profile of retinol from the lecithin-stabilised nanoparticle-coated capsule formulations described in Example 1 was undertaken using excised pig skin with Franz diffusion cells. The study was made in comparison with an unencapsulated (control) lecithin-stabilised emulsion of retinol in triglyceride oil. The skin from the abdominal area of a large white pig was separated and after removal of hair and the underlying fat layer, was kept at −80° C. until required. Skin samples were mounted to diffusion cells and 100 μl of the retinol formulation applied to achieve the thin layer on the skin sample surface, using 5 ml of water-ethanol 50-50 as a receptor medium. All experiments were carried out under occluded conditions.

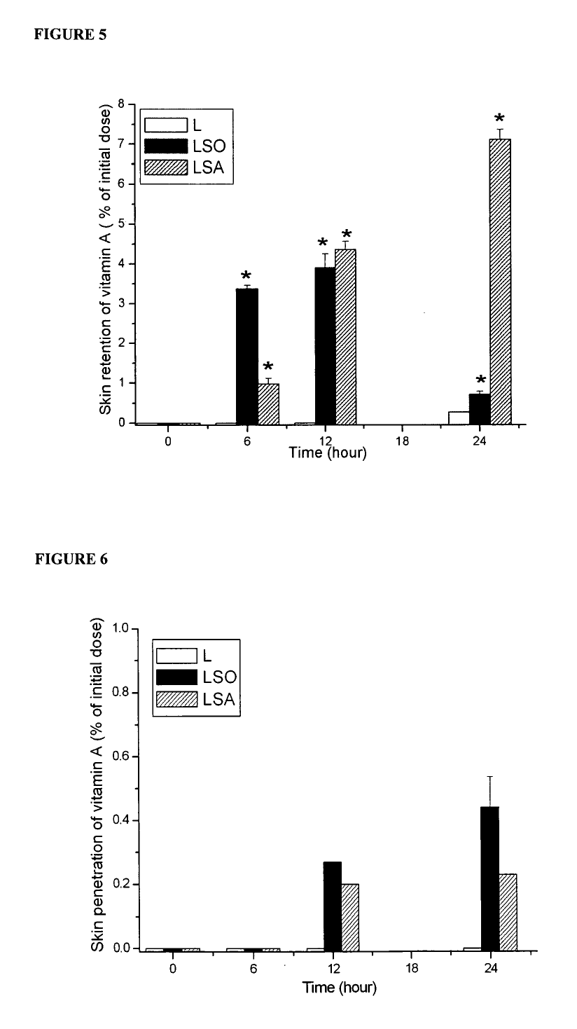

At 6, 12 and 24 hours, skin samples were taken and extracted with acetone to determine the concentration of retinol taken up and retained in the whole skin (epidermis, including stratum corneum, and dermis). In addition, at the completion of the experiment, 200 μL samples from receptor phase (ethanol-water 50-50) and skin surface were analysed with HPLC to quantify the amount retained in the skin (epidermis, including stratum corneum, and dermis) and the amount in the receptor phase (ie the amount that has penetrated through the whole thickness of the skin). The results are shown in FIGS. 5 and 6.

At all time points, the skin (epidermis, including stratum corneum, and dermis) retention of retinol was increased significantly for the nanoparticle-coated capsule formulations compared to unencapsulated control emulsions stabilised with lecithin. The results were statistically analysed with T test and ANOVA test and significance is marked in FIG. 5 with asterisks for P values less than 0.05.

The described retinol formulations, while being model formulations, may be used in topical skin application (eg for cosmetic purposes) wherein the “target layer” for the delivery of the retinol is the upper layers of skin (epidermis, including stratum corneum, and dermis). Transport across the skin into systemic blood circulation is undesirable in such application, and it simply leads to the “loss” of the active substance. Surprisingly, it was found that the amount of retinol detected in the receptor phase was negligible (FIG. 6) for the formulations (ie less than 0.5%). Thus, in vitro dermal delivery results using retinol as a model active substance show that negatively charged nanoparticle-coated capsule formulations efficiently deliver an active substance to the skin following topical application to the skin surface.

### b) Oleylamine-Stabilised Formulations (Positively Charged Capsules)

A study of the release profile of retinol from the oleylamine-stabilised nanoparticle-coated capsule formulations described in Example 1 was also undertaken using excised pig skin with Franz diffusion cells as described in Example 2 (a) above. In this case, the study was made in comparison with an unencapsulated (control) oleylamine-stabilised emulsion of retinol in triglyceride oil.

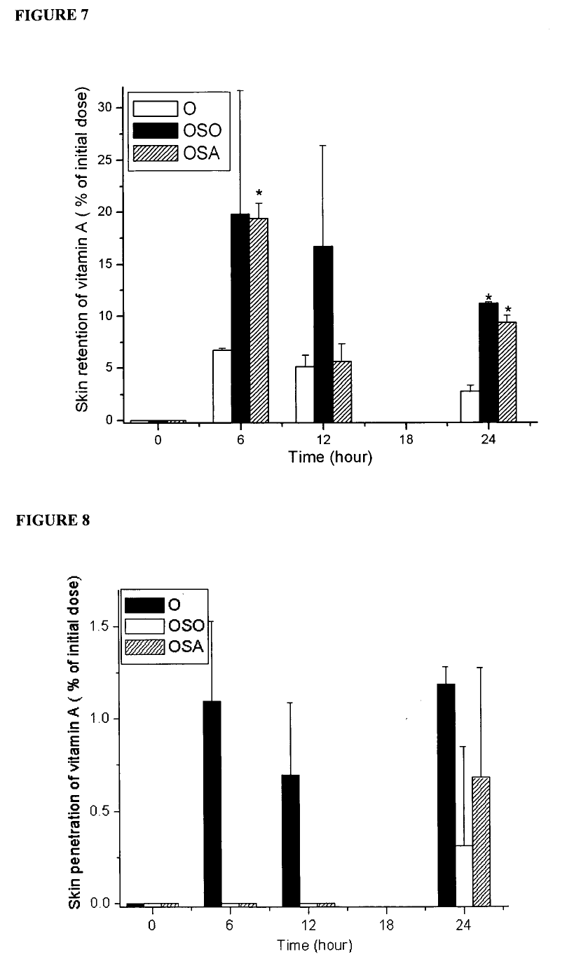

The results obtained with these positively charged emulsions (see FIG. 7) similarly showed enhancement in skin retention of retinol by nanoparticle encapsulation of the emulsion. Moreover, the oleyalmine-stabilised formulation generally showed higher skin retention compared to the lecithin-stabilised formulations tested in (a) above. Again, low levels (ie less than 1%) of the retinol penetrated through the skin and into the receptor phase (see FIG. 8). Thus, in vitro dermal delivery results using retinol as a model active substance show that positively charged nanoparticle-coated capsule formulations efficiently deliver an active substance to the skin following topical application to the skin surface.

c) Distribution of Vitamin A in Oleylamine-Stabilised Formulations into Different Skin Layers

Pig skin was mounted to Franz diffusion cells and treated with the oleylamine-stabilised nanoparticle-coated capsule formulations as described above. At 6 hr, the skin samples were removed from the Franz diffusion cells and frozen and sliced in 50 μm horizontal sections (TISSUE-TEK II, CRYOSTAT, MILES) and analysed for vitamin A (retinol) content with HPLC after extraction with acetone. According to light microscopy studies, the first 100 μm of the skin represents the stratum corneum and upper viable epidermis and the skin depth of between 100 and 200 μm mainly consists of viable epidermis; and the following sections represent the dermis of porcine skin (Jenning et al., 2000).

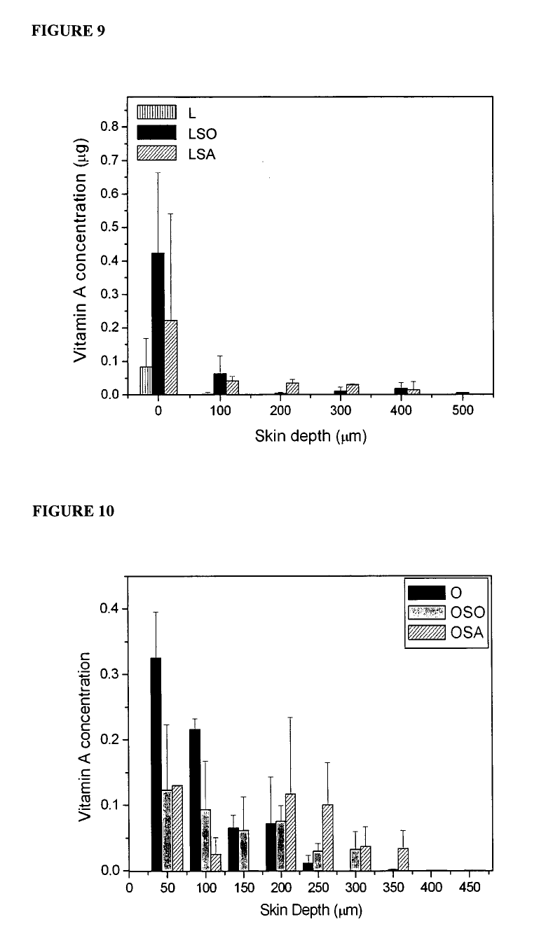

As presented in FIG. 9, for the oleylamine-stabilised control formulation (O), retinol is mostly accumulated in the stratum corneum with the maximum retinol concentration in the first 50 μm depth of skin. Meanwhile, the oleylamine-stabilised nanoparticle-coated (silica-in-oil) formulation (OSO) and the oleylamine-stabilised nanoparticle-coated (silica-in-aqueous phase) formulation (OSA) tended to show a more even distribution of retinol in the different skin layers with the maximum retinol concentration located in viable epidermis.

Accordingly, the kinetics of skin penetration and distribution were changed in the presence of silica nanoparticle layers, that is, the presence of silica nanoparticles was associated with higher delivery of retinol to target skin layers (viable epidermis and upper dermis).

Examples 1 and 2 show that retinol, a compound that has been difficult to formulate with traditional techniques, can be successfully encapsulated by nanoparticles to form a nanoparticle-coated capsule formulation. Further, this formulation protected the retinol from degradation following UV exposure, to which it is normally sensitive, and was capable of delivering the retinol to the skin. Other active substances of interest to the pharmaceutical, nutritional and cosmetic industries may be similarly formulated for dermal delivery.

### Example 3

### Depth Profile of Skin Penetration of Acridine Orange 10-Nonyl Bromide Containing Nanoparticle-Coated Capsule Formulations

Acridine orange 10-nonyl bromide is a lipophilic fluorescent dye and, accordingly, can be considered a lipophilic model drug compound. The present applicant investigated the depth of penetration of acridine orange 10-nonyl bromide when delivered by oleylamine or lecithin-stabilised nanoparticle-coated capsule formulations using excised pig skin with Franz diffusion cells.

### a) Preparation of Acridine Orange 10-Nonyl Bromide Formulations Stabilised by Lecithin

Lecithin (0.6 g) emulsifier and acridine orange 10-nonyl bromide (0.05 g) was dissolved in triglyceride oil (Miglyol 812™) (10 g), and then added to water (total sample weight: 100 g) for control emulsions, or to the silica dispersion described in step (c), to form capsules as described in step (d) below. In some experiments, the emulsifier, acridine orange 10-nonyl bromide and oil mixture was added to water and a portion of the water was replaced with the silica dispersion described in step (c), to form capsules as described in step (d). The resulting product was mixed using a high pressure homogeniser (5 cycles at 500 to 1000 bars).

### b) Preparation of Acridine Orange 10-Nonyl Bromide Formulations Stabilised by Oleylamine

Oleylamine (1 g) emulsifier and acridine orange 10-nonyl bromide (0.05 g) was dissolved in triglyceride oil (Miglyol 812™) (10 g), and then added to water (total sample weight: 100 g) for control emulsions, or to the silica dispersion described in step (c), to form capsules as described in step (d) below. In some experiments, the emulsifier, acridine orange 10-nonyl bromide and oil mixture was added to water and a portion of the water was replaced with the silica dispersion described in step (c), to form capsules as described in step (d) below. The resulting product was mixed using a high pressure homogeniser (5 cycles at 500 to 1000 bars).

### c) Preparation of Nanoparticles

An aqueous dispersion of fumed silica (Aerosil® 380) nanoparticles (1 wt %) (ie hydrophilic nanoparticles) were prepared by sonication over at least a one hour period.

### d) Capsule Formation

For emulsions containing silica nanoparticles initially included in the aqueous phase, capsules were formed when the nanoparticle dispersion of step (c) was separately mixed with either of the emulsions as described in step (a) and step (b).

### e) Alternative Preparation (Silica Nanoparticles in Oil)

Capsules were also formed in an analogous manner wherein the nanoparticles were initially included in the triglyceride oil from which the emulsion is formed. For example, lecithin-stabilised nanoparticle-coated fluorescent dye capsules similar to that described in (a) above (except that silica nanoparticles are added directly to the triglyceride oil) were prepared by dissolving lecithin (0.6 g) emulsifier in the triglyceride oil (Miglyol 812™) (10 g) to which fumed silica (Aerosil® 380) nanoparticles (1 wt %) were then added. Then, the fluorescent dye (acridine orange 10-nonyl bromide) was added and dissolved in the triglyceride oil mixture, followed by the addition of water (total sample weight: 100 g). An emulsion was formed using a high pressure homogeniser (5 cycles at 500 to 1000 bars).

Alternatively, oleylamine-stabilised nanoparticle-coated fluorescent dye capsules, similar to those described in (b) above (except that silica nanoparticles were added directly to the triglyceride oil) was prepared by dissolving oleylamine (1 g) emulsifier in the triglyceride oil (Miglyol 812™) (10 g), to which fumed silica (Aerosil® 380) nanoparticles (1 wt %) is then added. Then, the fluorescent dye (acridine orange 10-nonyl bromide) was dissolved in the triglyceride oil mixture and water added (total sample weight: 100 g). An emulsion was formed using a high pressure homogeniser (5 cycles at 500 to 1000 bars).

Accordingly, silica-encapsulated emulsions were prepared with incorporation of silica nanoparticles from either the oil phase (LSO, OSO) or aqueous phase (LSA, OSA) of the emulsions. Acridine orange 10-nonyl bromide (a lipophilic agent) was incorporated into the oil phase. Control formulations were medium chain triglyceride oil (Miglyol®812)-in-water emulsions with 10% volume fraction of the oil phase; these emulsions were initially stabilised with lecithin or oleylamine and prepared by high pressure homogenisation (EmulsiFlex-C5, Avestin® Inc.).

### f) Confocal Laser Scanning Electron Microscopy of Skin

To evaluate the depth profile of skin transport of acridine orange 10-nonyl bromide, skin samples were topically treated with the formulations and loaded onto Franz diffusion cells as described in Example 2. The skin samples were then sliced horizontally (Kryostat 1720, Leitz) and imaged using a confocal microscope (Leica SP5 spectral scanning confocal microscope). Digital images were collected at constant gain and offset parameters from the individual skin slices for their fluorescence intensity. In addition, Z-stack was used to scan the full thickness skin along the depth.

The extent of the distribution of the dye along the different layers of skin indicated different penetration profiles of the fluorescent dye (ie acridine orange 10-nonyl bromide) when incorporated into lecithin- or oleylamine-stabilised emulsions, and significantly higher fluorescence intensity was observed for silica-coated formulations compared to control formulations. The depth of skin penetration for the lecithin-stabilised formulations was approximately 69, 180 and 120 μm for control (L), silica-in-oil (LSO) and silica-in-aqueous phase (LSA) formulations, respectively, with the maximum fluorescence intensity observed in the upper layers of the skin. In comparison, the oleylamine-stabilised formulations penetrated deeper into the skin and had higher fluorescence intensity inside the skin.

Skin samples were also examined following vertical slicing. Essentially, full-thickness porcine skin was treated with the formulations and loaded onto Franz diffusion cells as described in Example 2. They were removed from the diffusion cells after three hours and completely washed with ethanol-water and then MilliQ water. The skin samples were immersed in Tissue-Tek® in plastic holders, and transferred to isopentane and then frozen in liquid nitrogen. Alternatively, the skin samples in the holders were incubated inside the Kryostst (Kryostat 1720, Leitz) until frozen. The frozen skin was sectioned using the Kryostat in 25 micrometer-thick sections perpendicular to epidermis and dermis. The samples were imaged using a confocal microscope as above.

In skin samples treated with the lecithin-stabilised control formulation (L), the fluorescent dye (ie acridine orange 10-nonyl bromide) only penetrated a few micrometers into stratum corneum which is the outermost layer of the skin. In skin samples treated with lecithin-stabilised formulations with silica-in-oil (LSO) or silica-in-aqueous phase (LSA), the fluorescent dye penetrated up to the stratum corneum and upper viable epidermis.

In skin samples treated with the oleylamine-stabilised control formulation (O), the fluorescent dye (ie acridine orange 10-nonyl bromide) accumulated in the stratum corneum; whereas in skin samples treated with oleylamine-stabilised formulations with silica-in-oil (OSO) or silica-in-aqueous phase (OSA), distribution of acridine orange 10-nonyl bromide was deeper, up to viable epidermis and upper dermis. Overall, the penetration was generally stronger for the oleylamine-stabilised formulations compared to the lecithin-stabilised formulations. This may be due to the electrostatic interactions between positively charged emulsion droplets and negatively charged skin lipids.

Thus, in accordance with the previous findings for retinol, the confocal images of the skin sections confirmed that the presence of silica nanoparticles in the formulations triggers the deeper distribution of the fluorescent probe within the skin into viable epidermis and upper dermis.

Encapsulation of emulsion droplets with silica nanoparticles offers better dermal delivery characteristics in favour of improved topical delivery of model lipophilic drugs. Higher skin uptake and deeper penetration of oleylamine-stabilised emulsions compared to lecithin-stabilised emulsions can be related to advantageous electrostatic interactions between positively charged emulsion droplets and negatively charged skin lipids.

Modifications and variations such as would be apparent to persons skilled in the art are deemed to be within the scope of the present invention. For example, although the invention is generally discussed with reference to emulsion droplets, the techniques discussed can generally be applied to liposomes, other vesicle systems and other similar vehicles.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

All publications mentioned in this specification are herein incorporated by reference. Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia or elsewhere before the priority date of each claim of this application.

### REFERENCES

1. Bos J. D. and Meinardi M. M. H. M., The 500 Dalton rule for the skin penetration of chemical compounds and drugs. Exp Dermatol. 9:165-169 (2000).
2. Brown M. B., et al., Dermal and Transdermal Drug Delivery Systems: Current and Future Prospects. Drug Delivery 13(3):175-187 (2006).
3. Elias P. M., Epidermal lipids, barrier function and desquamation. J. Invest. Dermatol. 80:44-49 (1983).
4. Jenning, V., et al., Vitamin A Loaded Solid Lipid Nanoparticles for Topical Use: Occlusive Properties and Drug Targeting to the Upper Skin. European Journal of Pharmaceutics and Biopharmaceutics 49(3): 211-218 (2000).
5. Scheuplein R. J. and Blank I. H. Permeability of the skin. Physiol. Rev. 51:702-747 (1971).

1. A method of dermal delivery of an active substance, said method comprising topically applying to the skin of a subject a formulation comprising droplets of a suitable carrier comprising said active substance and, optionally, an emulsifier, wherein said droplets are coated on their surface with at least one layer of nanoparticles, and wherein said active substance is not retinol or a retinol derivative.

2. The method of claim 1, wherein the active substance is delivered primarily to the dermis.

3. The method of claim 1, wherein the droplets comprise an oil-based or lipidic medium carrier.

4. The method of claim 1, wherein the droplets are coated with at least one layer of hydrophilic nanoparticles.

5. The method of claim 1, wherein the droplets are coated with at least one layer of hydrophobic nanoparticles.

6. The method of claim 1, wherein the nanoparticles are silica nanoparticles.

7. The method of claim 1, wherein the droplets are stabilised by an emulsifier.

8. The method of claim 7, wherein the emulsifier is selected from the group consisting of oleylamine, lecithin, sodium deoxycholate, 1,2-distearyl-sn-glycero-3-phosphatidyl ethanolamine-N, stearylamine and 1,2-dioleoyl-3-trimethylammonium-propane.

9. The method of claim 1, wherein the active substance is suitable for the treatment of a disease or condition which is localised, or at least partially localised, to the skin.

10. The method of claim 9, wherein the disease or condition is selected from the group consisting of skin cancer, psoriasis, eczema, bacterial and fungal infections, acne, dermatitis, inflammation, and rheumatoid arthritis.

11. A formulation for topical application to the skin, wherein said formulation comprises droplets of a suitable carrier comprising an active substance and, optionally, an emulsifier, wherein said droplets are coated on their surface with at least one layer of nanoparticles, and wherein said active substance is not retinol or a retinol derivative.

12. The formulation of claim 11, wherein the formulation releases the active substance in a sustained manner upon application to the skin.

13. The formulation of claim 11, wherein the formulation releases the active substance in a rapid manner upon application to the skin.

14. The formulation of claim 11, wherein the formulation is capable of releasing an active substance in a sustained manner upon application to skin, and wherein the formulation is produced by a method comprising the following steps:
(i) dispersing a discontinuous phase comprising a suitable carrier and an active substance into a continuous phase so as to form a two-phase liquid system comprising droplets of said discontinuous phase, each of said droplets having, at its surface, a phase interface; and
(ii) allowing nanoparticles provided to said two-phase liquid system to congregate at the phase interface to thereby coat said surface of the droplets in at least one layer of said nanoparticles;
wherein said two-phase liquid system is formed, or is otherwise adjusted, so as to have a concentration of a suitable electrolyte which enhances the nanoparticle congregation of step (ii) such that the coating on said surface of the droplets provided by the at least one layer of said nanoparticles presents a semi-permeable barrier to the active substance.

15. The formulation of claim 11, wherein the formulation is capable of releasing the active substance in a rapid manner upon application to skin, and wherein the formulation is produced by a method comprising the following steps:
(i) dispersing a discontinuous phase comprising a suitable carrier and an active substance into a continuous phase so as to form a two-phase liquid system comprising droplets of said discontinuous phase, and each of said droplets having, at its surface, a phase interface; and
(ii) allowing nanoparticles provided to said two-phase liquid system to congregate at the phase interface to thereby coat said surface of the droplets in at least one layer of said nanoparticles to form a nanoparticle-coated capsule formulation;
wherein the active substance is present in the discontinuous phase in an amount greater than its solubility limit in the discontinuous phase.

16. The formulation of claim 14, wherein the discontinuous phase comprises an oil-based or lipidic medium carrier and the continuous phase is aqueous.

17. The formulation of claim 11, wherein the droplets are coated with at least one layer of hydrophilic nanoparticles.

18. The formulation of claim 11, wherein the droplets are coated with at least one layer of hydrophobic nanoparticles.

19. The formulation of claim 11, wherein the nanoparticles are silica nanoparticles.

20. The formulation of claim 11, wherein the droplets are stabilised by an emulsifier.

21. The formulation of claim 20, wherein the emulsifier is selected from the group consisting of oleylamine, lecithin, sodium deoxycholate, 1,2-distearyl-sn-glycero-3-phosphatidyl ethanolamine-N, stearylamine and 1,2-dioleoyl-3-trimethylammonium-propane.

22. The formulation of claim 11, wherein the active substance is suitable for the treatment of a disease or condition which is localised, or at least partially localised, to the skin.

23. The formulation of claim 22, wherein the disease or condition is selected from the group consisting of skin cancer, psoriasis, eczema, bacterial and fungal infections, acne, dermatitis, inflammation, and rheumatoid arthritis.

24. The formulation of claim 15, wherein the discontinuous phase comprises an oil-based or lipidic medium carrier and the continuous phase is aqueous.
